(19) United States
(12) Patent Application Publication
(10) Pub. No.: US 2015/0217082 A1
(43) Pub. Date: Aug. 6, 2015

(54) SLEEP ASSISTANT SYSTEM, METHOD, AND NON-TRANSITORY COMPUTER-READABLE MEDIUM FOR ASSISTING IN EASING HARDSHIP OF FALLING ASLEEP

(71) Applicant: NATIONAL TAIWAN UNIVERSITY, TAIPEI (TW)

(72) Inventors: SHIH-CHUNG KANG, TAIPEI (TW); RAYLEIGN PING-YING CHIANG, TAIPEI CITY (TW); PETER LIU, TAIPEI (TW)

(21) Appl. No.: 14/319,604

(22) Filed: Jun. 30, 2014

Related U.S. Application Data
(63) Continuation-in-part of application No. 12/538,966, filed on Aug. 11, 2009, now abandoned.

(30) Foreign Application Priority Data
Aug. 14, 2008 (TW) 097131037

(51) Int. Cl. A61M 21/02

(52) U.S. Cl. CPC A61M 21/02; A61M 2021/0027

(57) ABSTRACT

Sleep assistant system and method for assisting in easing hardship of falling asleep are provided. The method includes the following. Data indicating at least one biosignal is received. A falling-asleep hardship index is determined based on the received data to indicate hardship of falling asleep for a user. Sleep guidance in visual form and/or audio form is provided, based on the falling-asleep hardship index, to assist the user before falling asleep in changing the falling-asleep hardship index for the user from a first state to a second state, wherein the sleep guidance is a portion of user interaction between the second user device and the user. The second state, different from the first state, indicates a less hardship for falling asleep in terms of the at least one biosignal from the user.

The application is a continuation-in-part of the application Ser. No. 12/538,966, the entirety of which is incorporated by reference herein.

### FIELD OF THE INVENTION

The present invention relates to a sleep assistant system and method, especially to a sleep assistant system and method for assisting in easing hardship of falling asleep.

### BACKGROUND OF THE INVENTION

About one third of the human lifetime is spent on sleeping. Thus, the sleep is quite important. A good life quality is usually built up with the good sleep quality. To improve the basic of the life quality should start at the improvement of the sleep quality. Unfortunately, according to the research findings, about 11.7% of Americans (i.e. about 32 million people) suffer from the problem of insomnolence or sleeplessness. The patients are widely distributed in different ages, sexes, races and social levels. The insomnolence affects the life quality largely. When the sleep is unbalanced, the physical and psychological conditions will be largely influenced, and even the family, job and social relationships are impacted as well. Therefore, it is necessary to solve the sleep problem and to do the research for building up high quality sleep environments.

Generally speaking, people easily fall asleep in more comfortable, safe, and familiar environment. One of the methods to solve the sleep problem is to build an “optimum sleep environment”. However, currently the developments of building the optimum sleep environment still focus on the development and the integration of the monitoring apparatus in the medical engineering to monitor various action indexes of the sleeper, e.g. brain wave, respiration, snoring sound, muscle tension, oximetric concentration, etc., which can be used by doctor to diagnose various sleep disorders and to evaluate the improving conditions before and after the treatment. Undoubtedly, the current society imminently requires an apparatus and method for providing appropriate sleep knowledge to the user and controlling and building good sleep environments.

### SUMMARY OF THE INVENTION

The present invention provides a sleep assistant system and a sleep assistant method for assisting in easing falling-asleep hardship.

In accordance with one aspect of the present invention, a sleep assistant method for a sleeper in an environment is provided. The method comprises monitoring a bio-condition of the sleeper to collect bioinformation of the sleeper; performing a sleep coach mode, which analyzes the bioinformation and provides a sleep knowledge to the sleeper based on the analyzed results; and performing a sleep environment adjusting mode, which adjusts the environment based on the bioinformation.

In an embodiment, the bioinformation includes at least one selected from a group consisting of a heartbeat, a body temperature, a blood pressure, a skin conductivity and a respiration rate.

In an embodiment, the sleep knowledge includes at least one selected from a group consisting of a direction from a doctor, a prescription by a doctor, a sleep medical knowledge, a medical treatment knowledge, a pre-sleep action and a sleep skill.

In accordance with another aspect of the present invention, a sleep environment adjusting system for a sleeper in an environment having an environment parameter is provided. The system comprises a sensor sensing the sleeper to obtain bioinformation; an environment adjusting device disposed in the environment; and an electronic processor electrically connected with the sensor and the environment adjusting device, and controlling the environment adjusting device based on the bioinformation.

In an embodiment, the sensor is disposed on the sleeper, and the electronic processor has a sleep environment adjusting mode controlling the environment adjusting device to adjust the environmental parameter based on the bioinformation and a preference of the sleeper.

In an embodiment, the environmental parameter comprises at least one selected from a group consisting of a visual effect, an acoustic volume, a temperature, a humidity and an air condition.

In an embodiment, the environment adjusting device comprises at least one selected from a group consisting of an air conditioner, an illuminating device, an audio device, a wake-up device and a combination thereof.

In an embodiment, the electronic processor is electrically connected with the sensor and the environment adjusting device via one of a wireless and a wired connections.

In an embodiment, the sleep environment adjusting mode is based on a fuzzy algorithm program.

In accordance with a further aspect of the present invention, a sleep coach apparatus for a sleeper is provided. The apparatus comprises a sensor sensing the sleeper to obtain bioinformation; and an electronic processor electrically connected with the sensor, and providing a sleep knowledge to the sleeper based on the bioinformation.

In an embodiment, the sensor is a biosensor, and the sleeper is one of a person ready to sleep and a person falling asleep, i.e. a user of the sleep assistant device.

In an embodiment, the bioinformation comprises at least one selected from a group consisting of a heartbeat, a body temperature, a blood pressure, a skin conductivity and a respiration rate.

In an embodiment, the electronic processor is one selected from a group consisting of a personal computer, a notebook computer and a server, and is electrically connected with the sensor via one of a wireless and a wired connections.

In an embodiment, the apparatus further comprises a display device electrically connected with the electronic processor.

In an embodiment, the electronic processor has a sleep coach mode analyzing the bioinformation and providing the sleep knowledge to the sleeper via the display device.

In an embodiment, the sleep coach mode comprises an intellectual algorithm program, which is designed based on at least one of a direction from a doctor and a prescription by a doctor.

In an embodiment, the display device comprises one selected from a group consisting of a cathode ray tube display, a liquid crystal display, a plasma display, a touch panel display and a projector.

In an embodiment, the sleep knowledge comprises at least one selected from a group consisting of a direction from a doctor, a prescription by a doctor, a sleep medical knowledge, a medical treatment knowledge, a pre-sleep action and a sleep skill.

In accordance with another aspect of the present invention, a sleep assistant system is provided, including a first user device and a second user device. The first user device includes at least one sensor for sensing at least one biosignal from a user. The second user device, which is able to communicate with the first user device to receive data indicating the at least one biosignal, is for providing, based on the received data, sleep guidance in visual form and/or audio form to assist the user before falling asleep in changing a falling-asleep hardship index for the user from a first state to a second state, wherein the sleep guidance is a portion of user interaction between the second user device and the user. The second user device determines the falling-asleep hardship index for the user based on the at least one biosignal; the falling-asleep hardship index determined before the sleep guidance is provided is at the first state. The falling-asleep hardship index determined after the sleep guidance is provided is at the second state. The second state, different from the first state, indicates a less hardship for falling asleep in terms of the at least one biosignal from the user.

In accordance with still another aspect of the present invention, an embodiment of a method for assisting a user in easing hardship of falling asleep is provided. The method includes the following. Data indicating at least one biosignal is received. A falling-asleep hardship index is determined based on the received data to indicate hardship of falling asleep for a user. Sleep guidance in visual form and/or audio form is provided, based on the falling-asleep hardship index, to assist the user before falling asleep in changing the falling-asleep hardship index for the user from a first state to a second state, wherein the sleep guidance is a portion of user interaction between the second user device and the user. The falling-asleep hardship index determined before the sleep guidance is provided is at the first state. The falling-asleep hardship index determined after the sleep guidance is provided is at the second state. The second state, different from the first state, indicates a less hardship for falling asleep in terms of the at least one biosignal from the user.

In accordance with still another aspect of the present invention, an embodiment of a non-transitory computer-readable medium, having stored thereon instructions, which when executed by a processor, cause the processor to perform: a method for assisting a user in easing hardship of falling asleep, as exemplified above.

### BRIEF DESCRIPTION OF THE DRAWINGS

These and other features, aspects, and advantages of the present invention will become better understood with regard to the following description and accompanying drawings as follows.

FIG. 1 is the schematic diagram showing the sleep assistant system according to an embodiment.

FIG. 2 is the schematic diagram showing the module configurations of the sleep assistant apparatus according to an embodiment.

FIG. 3 is a flow chart for performing the sleep assistant method according to an embodiment.

FIG. 4 is the schematic diagram showing an embodiment of the sleep assistant method.

FIG. 5 is a block diagram illustrating a sleep assistant system according to an embodiment.

FIG. 6 is a block diagram illustrating a sleep assistant system according to another embodiment.

FIG. 7 is a schematic diagram illustrating an embodiment of user interaction between the sleep assistant system and the user.

FIG. 8 is a schematic diagram illustrating an embodiment of a user interface of the sleep assistant system.

FIG. 9 is a block diagram illustrating a sleep assistant system according to still another embodiment.

FIG. 10 is a flowchart illustrating a sleep assistant method according to an embodiment.

FIGS. 11 and 12 are two embodiments of the sleep assistant method in FIG. 10.

### DETAILED DESCRIPTION OF THE INVENTION

The present invention will now be described more specifically with reference to the following embodiments. It is to be noted that the following descriptions of embodiments of this invention are presented herein for the purposes of illustration and description only; it is not intended to be exhaustive or to be limited to the precise form disclosed.

Embodiments of an apparatus and a method for building good sleep environments are proposed. The relevant factors influencing the sleep are quite diversified. Both the conditions of the sleeper and the environment are considered simultaneously, and make it complicated to build the appropriate sleep environment, since what each person recognizes and feels is different. Therefore, the optimum sleep environment would be different as the requirements of the sleep environment for each person are different. All the life pace, physical environments and psychological factors and so on would influence the quality of the sleep. The influence factors related to the life pace include the busy and tense modern life and the job time difference. The influence factors related to the physical environments include light, sound, temperature, air quality, bedding and pillow, etc. The influence factors related to the psychological factors include thinking and the psychological response to the stimulation of the environments. Usually all the above factors would influence the sleep quality of a person, and thus are taken into account and integrated into the embodiment of the sleep assistant system to build up the optimum sleep environments for a sleeper or a sleeper-centered bedroom.

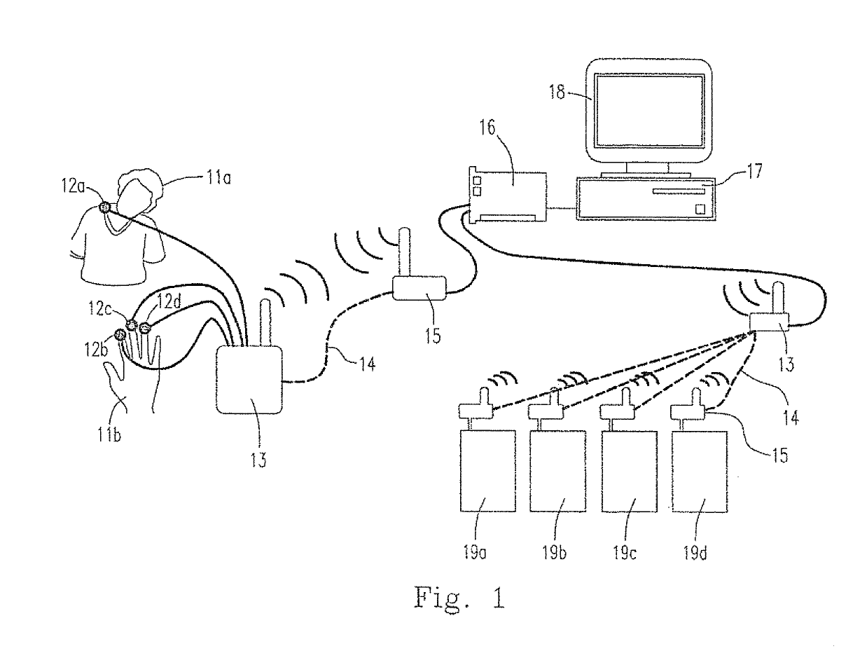

Please refer to FIG. 1, which shows the sleep assistant system according to an embodiment of the present invention. FIG. 1 shows a body 11a, a hand 11b of a sleeper, sensors 12a, 12b, 12c and 12d, front transceiver 13, data transmission cord 14, rear transceiver 15, data acquisition device 16, personal computer (PC) 17, display device 18, environment adjusting devices 19a, 19b, 19c and 19d, and the connection relationships among the above devices or elements. In this embodiment, the sleeper means a person ready to sleep or a person falling asleep, and the sleep means pre-sleep or during sleeping.

In this embodiment, the PC-based data acquisition (DAQ) configuration is adopted to collect and process various bioinformation data of the sleeper. However, the present invention is not limited to the PC-based data acquisition structure. Instead of the PC-based DAQ, any data acquisition configuration able to collect and process various bioinformation data of the sleeper can also be integrated into the embodiment of the sleep assistant system to reach the same effect of the embodiment.

The sensors 12a, 12b, 12c and 12d in this embodiment can be biosensors, and can be disposed in body 11a, hand 11b or other part of the body of the sleeper to collect/monitor/measure/sense the raw data of various bio-action indexes, including heartbeat, blood pressure, skin conductivity, respiration condition, etc. The monitoring of these bioinformations usually proceeds prior to sleep or during the sleep. These bioinformations can be used to check the sleep quality of the sleeper. The biosensors in the market or in the research can be integrated into the sleep assistant system of this embodiment of the present invention to acquire the monitored bioinformation of a sleeper prior to sleep or during the sleep. These monitored bioinformations can be extended to more knowledge in the sleep medicine field to further understand the sleep behavior of the human beings.

The signals of the bioinformations can be collected by the sensors 12a, 12b, 12c and 12d, and transmitted to front transceiver 13. These signals can be digital or analog signals depending on whether the inputs/outputs (I/O) of the sensors 12a, 12b, 12c and 12d are digital or analog. The front transceiver 13 and rear transceiver 15 can be wireless transceivers or wired transceivers. The front transceiver 13 transmits the signals of the bioinformations from the sensors 12a, 12b, 12c and 12d to the rear transceiver 15, and receives the signals or commands from the rear transceiver 15. When the wireless communication is adopted for the front and rear transceivers, various wireless communication techniques can be integrated into the sleep assistant system of the embodiment of the present invention, such as IEEE 802.11a (5 GHz), IEEE 802.11b/g (2.4 GHz), IEEE 802.11n (5 and/or 2.4 GHz), bluetooth (2.4 GHz), and Worldwide Interoperability for Microwave Access (WiMAX, 2.3, 2.5 and 3.5 GHz). Alternately, the front transceiver 13 can be connected with rear transceiver 15 by data transmission cord 14.

After the sensors 12a, 12b, 12c and 12d sense the bioinformations of the sleeper, the data acquisition device 16 acquires the raw data transmitted by rear transceiver 15, and transmits these data to PC 17 for the subsequent data processing or computation. In the PC-based configuration, data acquisition device 16 is a DAQ card. If the analog I/O configuration is adopted for the sensors, data acquisition device 16 can be an AD converter DAQ card to convert the acquired analog signals into the digital signals.

To sum up, in an embodiment of the present invention, the sensors 12a, 12b, 12c and 12d collect the raw data of the bioinformations, and data acquisition device 16 acquires and transmits these raw data to PC 17 for the subsequent data processing. In an embodiment, data acquisition device 16 is directly electrically connected with the sensors 12a, 12b, 12c and 12d to acquire the signals without passing through front transceiver 13 and rear transceiver 15.

Furthermore, in the sleep assistant system of an embodiment of the present invention, PC 17 is electrically connected with the display device 18. The PC 17 contains the software of the sleep coach mode and the sleep environment adjusting mode. In an embodiment of the present invention, the sleep coach mode and the sleep environment adjusting mode are set up in the LabView program in the PC-based configuration. After PC 17 receives the signals from data acquisition device 16, the sleep coach mode and the sleep environment adjusting mode can obtain various bioinformations from data acquisition device 16.

As an example, the sleep coach mode installed in PC 17 is implemented by an intellectual algorithm program or one or more program modules, designed based on the direction from a doctor or a prescription by a doctor, and can analyze the monitored bioinformations to obtain the indexes of the sleep quality, e.g. the relative percentage of each periods during the sleep, arousal index, cyclic alternating pattern (CAP), etc. Accordingly, it can be determined what kind of sleep knowledge is going to be provided to the sleeper. This sleep knowledge can include the direction from the doctor, the prescription by the doctor, the sleep medical knowledge, medical treatment knowledge, proper pre-sleep actions (i.e., preparation before sleeping), sleep skills, etc. After the sleep coach mode further analyzes the monitored bioinformations, the medical suggestions or prescriptions will be shown on the display device 18 of the PC 17 as the visual interface, which is a communication means between the sleep coach mode and the sleeper, and can be designed as an interactive way to response to the medical suggestions. After the sleep knowledge is transmitted to the sleeper via this clear and easy-understanding way, the physical and psychological conditions of the sleeper can be effectively changed, and the sleep quality of the sleeper can be gradually improved.

That is, after PC 17 receives the signals from data acquisition device 16, the sleep coach mode analyzes these signals of the bioinformations, and then display device 18 provides the appropriate and helpful sleep knowledge to the sleeper. Here the display device 18 can be a cathode ray tube (CRT) display, a liquid crystal display (LCD), a plasma display, a touch panel display or a projector.

The sleep environment adjusting mode installed in PC 17 can be a program based on a fuzzy algorithm, and can adjust the environment where the sleeper is located based on the monitored bioinformations. The sleep environment adjusting mode receives the bioinformation from the sleeper, then determines the relative important levels of each bioinformation, and then decides how to adjust various sleep quality influence factors, including the sound, light, room temperature, air condition, etc. The sleep environment adjusting mode can be further integrated with the architectural technique to control the visual and audio effects so as to build up the good sleep environment. Then the environment factors can be specifically tailored for the individual sleeper to build up the appropriate sleep environment to improve the sleep quality and to effectively assist the treatment for the sleep disorder.

The environment adjusting devices 19a, 19b, 19c and 19d can be the air conditioner, illumination device, audio device and wake-up device. The air conditioner can be the window air conditioner, separated air conditioner or central air conditioner. The illumination device can be a fluorescent lamp, a desk lamp, a stand lamp or a bed lamp. The wake-up device can be an alarm clock.

When PC 17 receives the signals from data acquisition device 16, the sleep environment adjusting mode analyzes these signal of the bioinformations, and the analyzed results are transmitted to rear transceiver 15 via data acquisition device 16 and front transceiver 13 to control the environment adjusting device 19a, 19b, 19c and 19d so as to adjust the environment factors. In an embodiment of the present invention, the analyzed results from the data acquisition device 16 can be used to directly control the environment adjusting devices 19a, 19b, 19c and 19d without pass front transceiver 13 and rear transceiver 15.

Basically, a sleep coach apparatus can include the sensors 12a, 12b, 12c and 12d, front transceiver 13, cord 14, rear transceiver 15, data acquisition device 16, PC 17 and display device 18. The sleep environment adjusting system can include the sensors 12a, 12b, 12c and 12d, front transceiver 13, cord 14, rear transceiver 15, data acquisition device 16, PC 17, display device 18 and environment adjusting devices 19a, 19b, 19c and 19d.

The above sleep assistant system can be set up in any space, including the bedrooms, hotels, dormitories or hospitals.

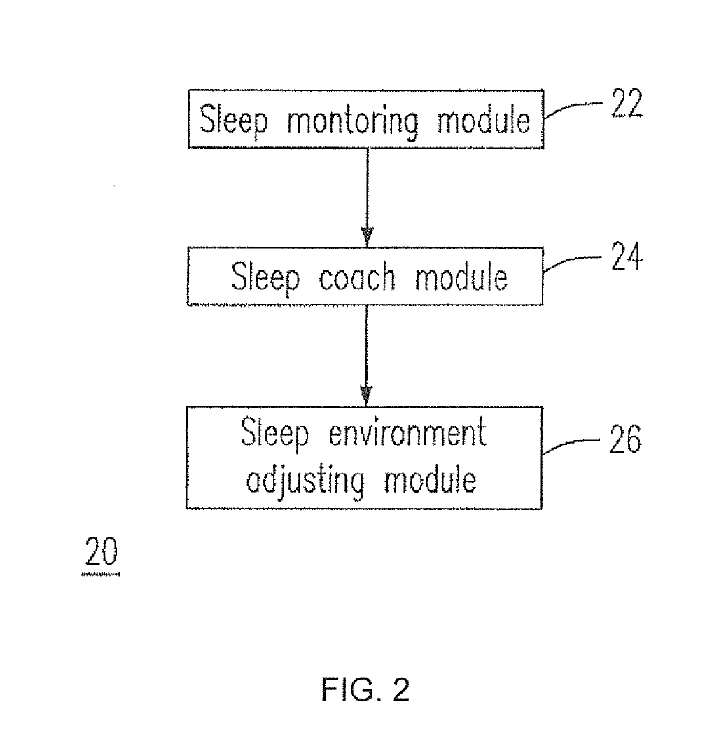

The sleep assistant system of the above embodiments of the present invention can be further divided into several modules for facilitating the modulized implementations according to the different functions for each stage. Please refer to FIG. 2, which shows the module configurations of the sleep assistant apparatus of the above embodiments of the present invention. The sleep assistant apparatus 20 in FIG. 2 includes the sleeper monitoring module 22, sleep coach module 24 and sleep environment adjusting module 26.

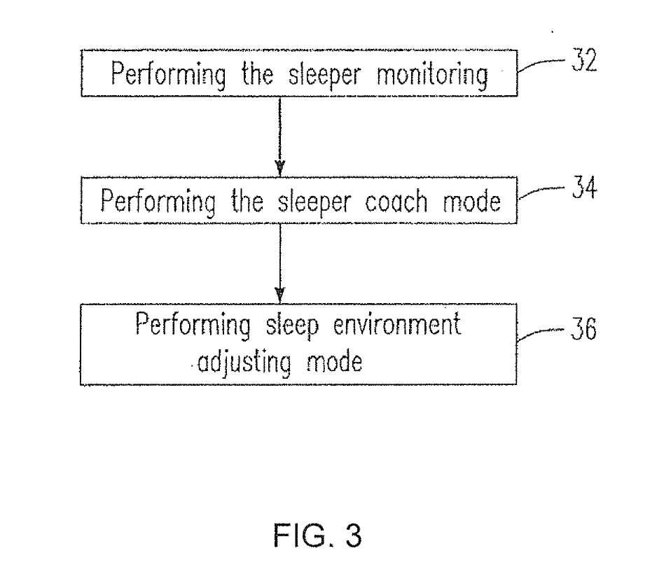

To sum up the above mentioned concepts of the above embodiments of the present invention, a sleep assistant method can be obtained. Please refer to FIG. 3, which shows the flow chart for performing the sleep assistant method of the above embodiments of the present invention. The method in FIG. 3 includes the steps of performing the sleeper monitoring 32, performing the sleeper coach mode 34, and performing the sleep environment adjusting mode 36.

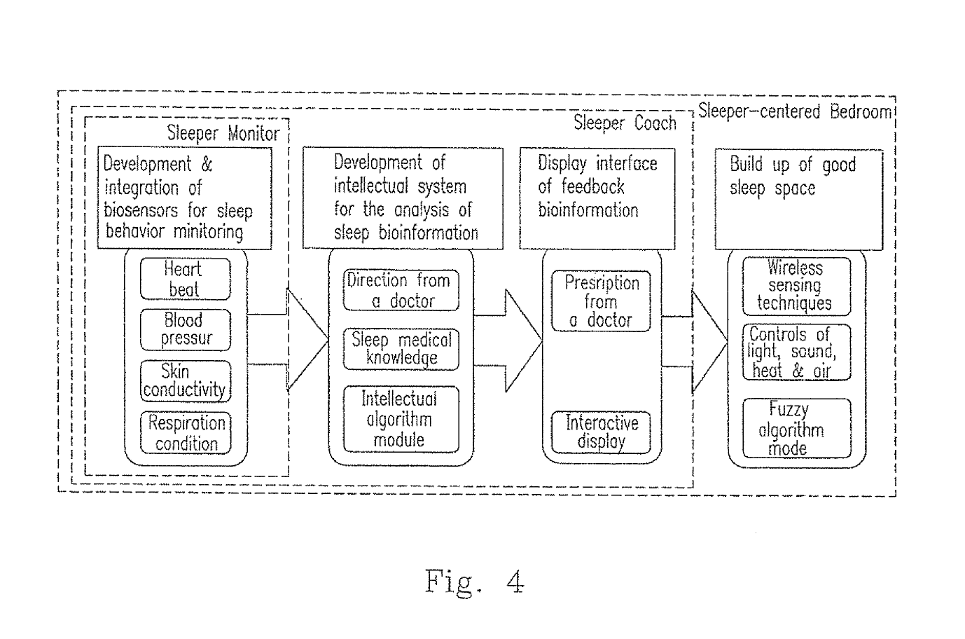

From the above, the clear concepts shown in FIG. 4 can be obtained. Please refer to FIG. 4, which shows the concepts of the sleep assistant method of the above embodiments of the present invention.

As such, the sleep assistant apparatus and the sleep assistant method according to the above embodiments of the present invention are based on the economic and portable sleep monitoring device. The biosensors are developed and integrated into the monitoring of the sleep behaviors. The conditions of the sleeper are sensed by the biosensors with the portability or easy installation so as to build up the optimum sleep environment. The sleep monitoring devices are used to monitor various bio-action indexes during the sleep, e.g. heartbeat, body temperature, skin conductivity, respiration rate, etc. For the patients with the sleep disorder, these bio-action indexes can be used for the doctor's diagnosis and the comparison of the conditions before and after the treatments. Further cooperating with the other sensing devices in the intellectual space, an integrated caring network can be built. The monitored indexes before and during the sleep can be further analyzed as the reference for the subsequent diagnosis.

In the following, further embodiments of sleep assistant system and method thereof are provided.

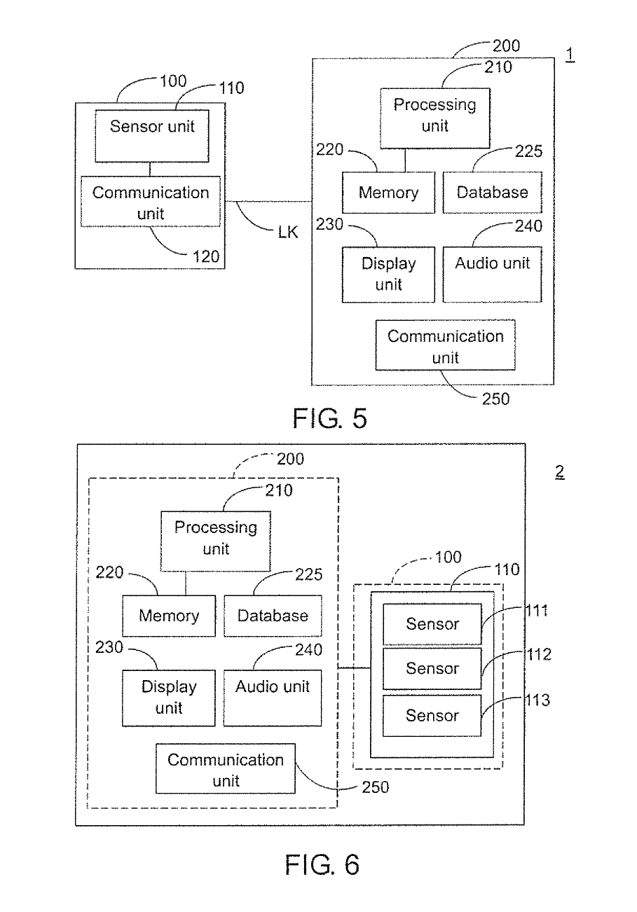

FIG. 5 is a block diagram illustrating a sleep assistant system according to an embodiment. In FIG. 5, a sleep assistant system 1 includes a first user device 100 and a second user device 200. The first user device 100 includes at least one sensor for sensing at least one biosignal from a user. The second user device 200 is able to communicate with the first user device 100 to receive data indicating the at least one biosignal. For example, the at least one biosignal includes one or more of a heartbeat signal, a body temperature signal, a blood pressure signal, a respiration rate signal, and a skin conductivity signal, without being limited thereto.

According to the embodiment, the second user device 200 provides, based on the received data, sleep guidance in visual form and/or audio form to assist the user before falling asleep in changing a falling-asleep hardship index for the user from a first state to a second state, wherein the sleep guidance is a portion of user interaction between the second user device and the user. The second user device 200 determines the falling-asleep hardship index for the user based on the at least one biosignal. For example, the falling-asleep hardship index determined before the sleep guidance is provided is at the first state; the falling-asleep hardship index determined after the sleep guidance is provided is at the second state; and the second state, different from the first state, indicates a less hardship for falling asleep in terms of the at least one biosignal from the user.

In an example, the first user device 100 is a wearable device able to communicate with the second user device 200 electrically or wirelessly. The wearable device, for example, is an electronic device which is wearable and may be realized in various forms, without limited to, such as a watch, a ring, clothes, shoes, a pair of glass, and so on. In another example, the first user device 100 can be implemented as a peripheral device of the second user device 200. The second user device 200 can be an electronic computing device, such as a mobile device, or such as a desktop computer system. The mobile device can be, but without limited to, for example, a smart phone, a tablet computer, and a notebook computer. In some examples as shown in FIG. 6, the sleep assistant system 1 can be implemented as a dedicated apparatus (e.g., a wearable device, a mobile device, or a computer) includes the first user device 100 and the second user device 200, wherein the first user device 100 having one or more sensor (e.g., sensors 111-113) for contacting with fingers (e.g., index finger, middle finger, and ring finger) of the user's hand is embedded into the dedicated apparatus. For example, the sensors would be bioelectrodes which detect galvanic skin resistance, oxygen saturation (SaO2) or heartbeat rate.

Referring to FIG. 5, the first user device 100, for example, includes a sensor module 110 and a communication module 120. The sensor module 110 can include one or more sensors for sensing one or more biosignals of a user, wherein one sensor may be in contact with a suitable portion of the body of the user, such as a finger or hand or head or any part of the human body. The second user device 200, for example, includes a processing unit 210, a memory 220, a database 225, a display unit 230 (such as LCD or touch panel), an audio unit 240, and a communication unit 250 (such as circuit for data and/or mobile communication). The first user device 100 is capable of communicating with the second user device 200 with a communication link LK, e.g., electrically or wirelessly, without being limited thereto. In FIG. 6, a sleep assistant system 2 is a dedicated apparatus including the first user device 100 and the second user device 200, wherein the first user device 100 is embedded into the dedicated apparatus 2.

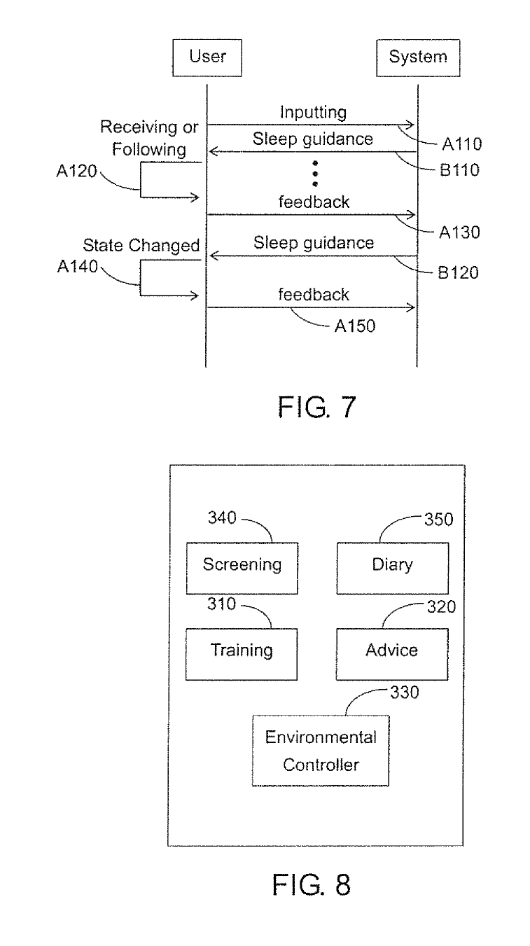

Referring to FIG. 7, the user interaction between the sleep assistant system and the user is illustrated in an example. In FIG. 7, as indicated in A110, data indicative of one's biosignal is inputted, and/or the user may input some information and instruction onto the sleep assistant system through the second user device. As indicated in B110, the sleep assistant system provides sleep guidance in response. The user then receives an advice included in the sleep guidance or follows one or more directions included in the sleep guidance, as indicated in A120. The sleep guidance is provided in visual form and/or audio form, such as images, video(s), voice, or music, or multimedia, to assist the user before falling asleep in changing a falling-asleep hardship index for the user from a first state to a second state, as indicated in A140. As illustrated in FIG. 7, the sleep guidance is a portion of the user interaction between the second user device and the user. During the sleep guidance or before two different pieces of sleep guidance, the user can feed back to the system, as indicated by A130 or A150, for example, by way of one or more biosignals or user response through the user interface of the system (e.g., by the first user device or second user device). In this way, biofeedback may be realized for the sleep assistant system to improve the sleep guidance.

Referring to FIG. 8, a user interface of the sleep assistant system, for example, through the second user device 200 is illustrated in an embodiment. FIG. 8 shows an example of the user interface of the sleep assistant system, with some graphical icons on a screen of the second user device 200, such as the graphical buttons labeled “Training” 310, “Advice” 320, “Environmental Controller” 330. The user may select one of the buttons from the screen so as to start the corresponding user interaction or program associated with each button. For example, “Training” button is selected and another screen shows one or more training courses for selection, for example, a respiration training and a meditation training, for easing one's difficulty of falling asleep. In another example, the user may select “Advice” button to obtain information about sleep, such as sleep hygiene, medical suggestion about using sleep pills. In other example, the user may select “Environmental Controller” button to have the environment condition for the user changed, wherein the environmental condition includes, but not limited to, one or more of light condition, sound condition, temperature condition, and air quality condition. In another embodiment, the user interface may include a “Screening” button 340 for associated with a screening program for inputting basic information about one's sleep and a “Diary” button 350 for a program for a user to record one's behavior before sleep. For example, the screening program can be implemented according to some standard, such as Pittsburgh Sleep Quality Index (PSQI) or 36-item Short Form (SF-36). In addition, the survey information may be used in the determination of sleep guidance and/or environmental condition control.

In one embodiment, the sleep guidance including training courses and/or advice can be provided based on the received data indicating at least one biosignal from the first user device. In addition, in accompany with the sleep guidance, the second user device 200 sends at least one control signal, based on the received data, to adjust environmental condition for the user so as to assist the user before falling asleep in changing the falling-asleep hardship index to a state indicates a less hardship for falling asleep in terms of the at least one biosignal from the user. Embodiments of sleep guidance and/or environmental condition control will be provided later for easing hardship of falling asleep with respect to a user.

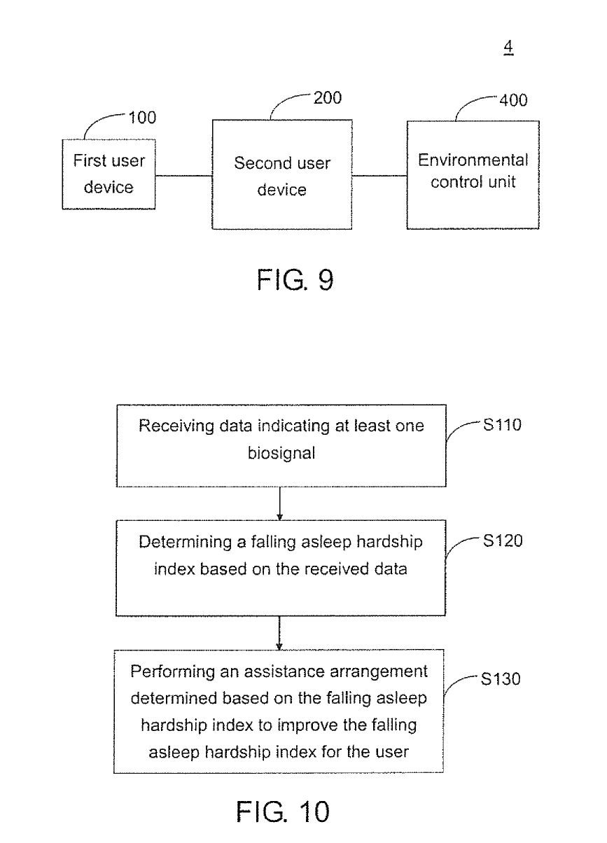

Referring to FIG. 9, another embodiment of a sleep assistant system is illustrated. In FIG. 9, a sleep assistant system 4 includes the first user device 100, the second user device 200, and an environmental control unit 400. For example, the environmental control unit 400 includes a plurality of environment adjusting devices operative based on the at least one control signal to adjust the environmental condition for the user.

FIG. 10 illustrates a sleep assistant method according to an embodiment. The sleep assistant method in FIG. 10, for example, may be used by a user device (e.g., the second user device 200) of a sleep assistant system according to the above embodiment. As indicated in step S110, data indicating at least one biosignal is received by the user device. As illustrated in step S120, a falling-asleep hardship index is determined based on the received data to indicate hardship of falling asleep for a user. As shown in step S130, an assistance arrangement determined based on the falling-asleep hardship index is performed to improve the falling-asleep hardship index for the user. The assistance arrangement indicates a selection of one or more types of sleep guidance and/or environmental condition control, based on the falling-asleep hardship index.

Various ways of implementation for each step of the method illustrated in FIG. 10 are provided as follows.

In step S110, one or more biosignal that may be associated with mental stress levels or anxiety levels of human being, such as biosignals indicative of heartbeat rate, respiration rate, body temperature, blood pressure, and skin conductivity level, without being limited thereto.

In step S120, the falling-asleep hardship index is determined based on the at least one biosignal to indicate the hardship of falling asleep for a user. For example, heart rate variability (HRV) can be used to indicate a stress level from nervousness to easiness and can be determined by the measurement of one's heartbeat rates over a period of time. The HRV can be determined by the first user device 100 or the second user device 200 based on the heartbeat rates over a period of time. Different approaches under time-domain and frequency-domain analysis for HRV can be applied in the determination of the falling-asleep hardship index.

In one example, Standard Deviation of Normal to Normal intervals (SDNN) for heart rates, regarded as time-domain indication of HRV, is used. The SDNN can be defined by

$$SDNN = \sqrt{\frac{1}{N} {\sum }_{N} {( {RR}_{i} - \overline{RR} )}^{2}} , \\ where$$

$$\overline{RR} = \frac{1}{N} {\sum }_{N} {RR}_{i}$$

and R is a point corresponding to the peak of the QRS complex of the electrocardiogram (ECG) wave; and RRᵢ is the interval between successive R points; and the time of measurement for calculation of SDNN may be a few minutes (e.g., 5 min) to hours (e.g., 24 hours). In general, the SDNN is related to the age of human beings and the normal SDNN decreases as the age increases. For example, the normal SDNN of a person at the ages of 10, 40, 60, and 70 are about 32, 25, 20, and 17 ms, respectively. When the SDNN of a user after doing something is determined to be lower than the normal SDNN for one's age or to be lower than the SDNN that was tested before, it can be determined that the user is under pressure. Conversely, when the SDNN of a user is higher than the normal, the user is not under pressure. In an example, the falling-asleep hardship index with respect to SDNN for a 40-year-old may be defined as in Table 1 as below (where FAHI, without limited to, is set to be in the range from 1 to 10, for example).

TABLE 1

| Falling-asleep | | |
|---|---|---|
| hardship index (FAHI) | SDNN (ms) | Indication |
| 1 | Above 25 | lowest stress level |
| 2 | 26 | |
| . . . | . . . | . . . |
| 7 | 20 | Immediate stress level |
| . . . | . . . | . . . |
| 10 | 15 or below | High stress levels |

In another example, frequency-domain analysis for HRV is taken. By signal processing technique such as Fast Fourier transformation (FFT) (or other transformation, such as Hilbert transform, Hilbert-Huang Transform), HRV can be further represented in terms of frequencies, e.g. categorized into high frequencies (HF), low frequencies (LF), and very low frequencies (VLF), wherein it can be defined that VLF≦0.04 Hz, 0.04≦LF≦0.15 Hz, 0.15≦HF≦0.4 Hz. In general, the HF components are associated with relaxation while the LF components are associated with stress, and negative emotions such as panic, depression, anxiety, and hostility have all demonstrated reduced HRV. For example, according to Dishman R K et al., “Heart rate variability, trait anxiety, and perceived stress among physically fit men and women,” Int J Psychophysiol. 2000 August; 37(2):121-33, HRV datasets were decomposed into low-frequency (LF; 0.05-0.15 Hz) and high-frequency (HF; 0.15-0.5 Hz) components using spectral analysis, and there was an inverse relationship between perceived emotional stress during the past week and the normalized HF component of HRV (P=0.038). Accordingly, the falling-asleep hardship index can be defined on the basis of the high frequency components, for example, as illustrated in Table 2 below, wherein TH1 to TH10 are decreasing values, indicating HF thresholds for determining the corresponding indexes, wherein TH10 is the lowest value among TH1 to TH10.

TABLE 2

| Falling-asleep | | |
|---|---|---|
| hardship index (FAHI) | HRV (HF) | Indication |
| 1 | TH1 | lowest stress level |
| 2 | TH2 | |
| 3 | TH3 | |
| . . . | . . . | . . . |
| 10 | TH10 | High stress levels |

In another example, the falling-asleep hardship index (FAHI) can be defined with a fewer number of HF thresholds (e.g., FAH=10 if TH10≦HF<TH6; FAHI=5 if TH6≦HF<TH3; and FAHI=1 if TH3≦HF≦TH1).

In addition, one or more type of biosignals then can be used as the basis of the falling-asleep hardship index. For example, respiration rate of a lower rate indicates a type of relaxation and can be associated with a FAHI of lower value (e.g., 1) while respiration rate of a higher rate indicates a type of nervousness or excited state and can be associated with a FAHI of higher value (e.g., 6). The relationship between respiration rate and the FAHI can be expressed as φ=w₂·X₂, wherein w₂ is a weight for index of respiration rate (X₂).

In another example, skin conductance could also be used for indication of psychological or physiological arousal. If the sympathetic branch of the autonomic nervous system is highly aroused, then sweat gland activity also increases, which in turn increases skin conductance. The combined changes between galvanic skin resistance and galvanic skin potential make up the galvanic skin response. Galvanic skin resistance (GSR) with a higher value indicates a type of relaxation and can be associated with a FAHI of lower value (e.g., 2) while GSR of a low value indicates a type of nervousness and can be associated with a FAHI of higher value (e.g., 8). The relationship between skin conductivity level and the FAHI can be expressed as φ=w₃·X₃, wherein w₃ is a weight for index of GSR (X₃), X₃=1/Y₃, Y₃ indicates GSR).

For example, the relationship between the biosignal and the falling-asleep hardship index can be realized in a look-up table form and stored in a database. Further, in some examples, a falling-asleep hardship index can be determined based on two or more biosignals, for example, HRV with skin conductivity level or respiration rate, by a relationship of weighting summation or other relationship. In an example, falling-asleep hardship index φ can be defined by:

φ=w₁·X₁+w₂·X₂+ . . . +wₙ·Xₙ, (Equation 1)

wherein wₖ is a weight for an index Xₖ based on one biosignal, 1≦k≦n, n≧2.

Further, in other example, one or more weights in Equation 1 can be changed based on one or more factors, e.g., from user subjective information and/or environmental condition(s). In some example, user subjective information may be data from the screening. For instance, user subjective information may be user's activity and/or habit or behavior before going to bed that can be the factors for changing the weight(s) for particular index(es) so as to indicate the degree of importance for the corresponding index. Specifically, the weight for some index (e.g., corresponding to HRV, respiration rate, or GSR) with respect to the user is increased before a specified time period (e.g., 30 minutes or one hour) of the time going to sleep (e.g., before 10:30 pm) when the sleep assistant system is informed that (e.g., by way of user input data or screening) the user has the habit of doing exercises at some time (e.g., 9:00 pm for one hour) or that the user has to work overtime for a period of time (e.g., the month) (i.e., life will be under pressure over the period of time). Moreover, the environmental condition(s) can also be the factor(s) for changing the weight(s). For example, if the room temperature is over a threshold (e.g., 30° C. or above), the index for HVR or GSR may be increased. For example, sensors, such as temperature sensor, humidity sensor, or light sensor, and so on, can be embedded in the second user device or can be disposed in the environment, or related signals can be provided by the environmental adjustment devices (e.g., air conditioner(s), lighting device(s), or audio device(s), in order for the second user device to determine the falling-asleep hardship index for the user.

In other examples, the falling-asleep hardship index may be determined based on the biosignal(s) and at least one sleep-parameter, such as Sleep Latency (SL), Sleep Period Time (SPT), Total Sleep Time (TST), Sleep Efficiency (SE), Slow Wave Sleep (SWS), Intermittent Time Awake/Wakefulness After Sleep Onset (LTA), Episodes of Wake (WE) and the number of Micro-Arousals. For example, if the sleep latency for a specific user is longer than 30 minutes, which may be known by the user device through user input or screening or by EEG (electroencephalography) detection from the user device, the falling-asleep hardship index for that user may be set a falling-asleep hardship index of a higher index (e.g., 8).

In step S130, an assistance arrangement determined based on the falling-asleep hardship index is performed to improve the falling-asleep hardship index for the user. The assistance arrangement indicates a selection of one or more types of sleep guidance and/or environmental condition control, based on the falling-asleep hardship index.

For example, the user device determines a type of sleep guidance to assist the user before falling asleep in changing his falling-asleep hardship index from a first state to a second state. In some example, the assistance arrangement can include additional environmental adjustment in order to facilitate easing the hardness of falling asleep. For example, Table 3 illustrates examples of different assistance arrangements determined based on the falling-asleep hardship index.

TABLE 3

| FAHI | Assistance arrangement | Indication |
|---|---|---|
| 1 | N/A | Least stress level |
| 2-4 | Advice (AD), environmental adjustment | Slight stress level |
| | (EA) | |
| 5-7 | Training (TR), EA | Middle stress level |
| 8-10 | TR, AD, EA | Higher stress levels |

In an embodiment, step S120 includes providing, based on the falling-asleep hardship index, sleep guidance in visual form and/or audio form to assist the user before falling asleep in changing the falling-asleep hardship index for the user from a first state to a second state, wherein the sleep guidance is a portion of user interaction between the second user device and the user. The falling-asleep hardship index determined before the sleep guidance is provided is at the first state; the falling-asleep hardship index determined after the sleep guidance is provided is at the second state; and the second state, different from the first state, indicates a less hardship for falling asleep in terms of the at least one biosignal from the user.

In an embodiment, the sleep guidance is determined based on the first state and includes directions (such as in a training course (TR)) in visual form and/or audio form to instruct the user to follow the directions to adjust one's respiration rate before falling asleep. For example, the training course may be deep-breathing exercises, with long exhalations. Specifically, the directions of the training course include slow inspiration, deep inspiration, breathing hold for 2-3 seconds, slow relaxed exhalation, and 5-10 times every hour. In another example, the directions may be rhythmic breathing.

In an embodiment, the sleep guidance is determined based on the first state and includes directions (such as in a training course (TR)) in visual form and/or audio form to instruct the user to follow the directions to practice meditation before falling asleep. For example, the training course may be daily sitting meditation practice, the directions of which includes an attentional focus on the breath and body-related sensations for 20-30 min per day. The practice of meditation can lead to changes in alpha wave behavior, which in turn may lead to quell anxiety and promote serenity.

In an embodiment, the sleep guidance is determined based on the first state and includes an advice (AD) about sleep, wherein the advice is presented in visual form and/or audio before falling asleep. In some examples, one or more advices of sleep hygiene can be provided, for example, “allowing enough time for sleep (e.g., 7-9 hours of sleep each day)”; “avoiding eating too much and alcohol before sleep and reducing intake of caffeine and other stimulants several hours before bedtime”; “exercising for twenty to thirty minutes or so five to six hours before sleep, but not immediately before sleep”; “seeking assistance from doctors or professionals for continuing difficulties with sleep, since specific sleep disorders may require particular treatments.” The advice may be provided through text, voice, image, video, or interactive operations. In an additional example, the advice related to environmental adjustment such as “arranging a sleep environment that is dark, quiet, and cool to help falling asleep quickly” can be provided and/or performed automatically by the sleep assistant system.

Environmental adjustment can be performed by the sleep assistant system. In an embodiment, at least one control signal, based on the received data, is sent to adjust environmental condition for the user so as to assist the user before falling asleep in changing the falling-asleep hardship index to a state indicates a less hardship for falling asleep in terms of the at least one biosignal from the user. In a scenario, for example, a user uses the sleep assistant system, before going to sleep, and the second user device initially determines a falling-asleep hardship index to be 7. Before the time of going to bed set in the second user device (e.g., 1 hour), the air conditioner is adjusted based on one control signal to reduce the room temperature from 30 to 27° C., and the lighting devices are adjusted to a lower brightness for preparation of going to bed, according to at least one control signal. In addition, the television or audio system may be set to a lower or gradually reducing volume, based on at least one control signal, before 30 minutes, for example, of the time for going to sleep. The above environmental adjustment(s) may be occurred before, after, or while the sleep guidance provided by the second user device. In some example, the second user device may be a smart device (such as mobile devices, or wearable devices, or the smart television, or smart home system). That is, the sleep guidance of the sleep assistant system may be integrated with or into the home automation or smart home technology (or system) to enhance the performance of easing the falling-asleep hardship for the user.

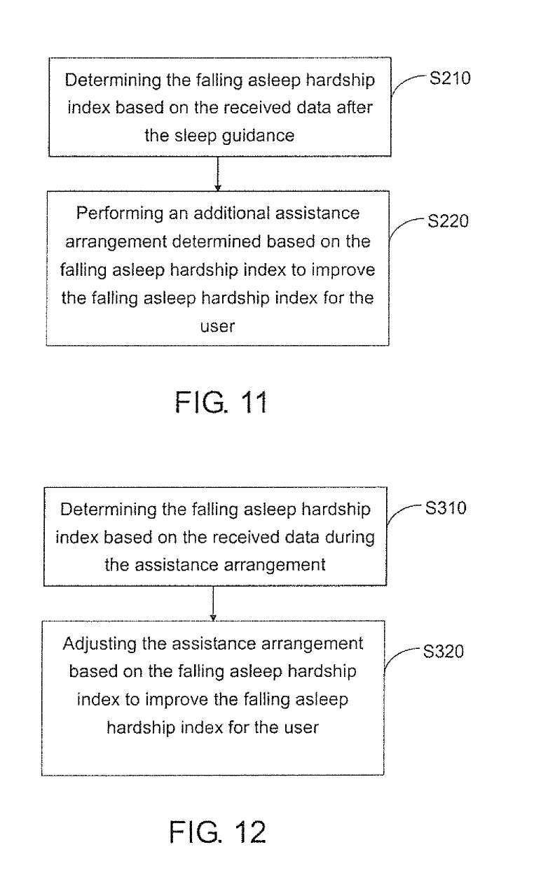

In some embodiments, the method can further provide additional sleep guidance according to feedback of the user's one or more biosignals (e.g., based on the state of falling-asleep hardship index after the sleep guidance). Referring to FIG. 11, an embodiment of the method in FIG. 10 is illustrated. As shown in step S210, the falling asleep hardship index is determined based on the received data after the sleep guidance. As illustrated in step S220, an additional assistance arrangement determined based on the falling-asleep hardship index at the second state is performed to improve the falling asleep hardship index for the user. For example, it is supposed that the falling-asleep hardship index is changed from 8.5 to 6.5 after the sleep guidance, and an additional assistance arrangement including an additional sleep guidance and environmental adjustment can then be provided according to step S220 as well as Table 3. In another embodiment, the additional assistance arrangement can be determined based on the data received during the previous sleep guidance.

In some embodiments, the method can further adjust the sleep guidance being performed according to feedback of the user's one or more biosignals. Referring to FIG. 12, an embodiment of the method in FIG. 10 is illustrated. As shown in step S310, the falling asleep hardship index is determined based on the received data during the assistance arrangement. As illustrated in step S320, the assistance arrangement is adjusted based on the falling asleep hardship index to improve the falling asleep hardship index for the user.

Furthermore, other embodiments further disclose a non-transitory computer or computing device readable information storage medium for storing program code or one or multiple program modules. In general, routines executed to implement the embodiments of the invention, may be implemented as part of an operating system or a specific application, component, program, object, module or sequence of instructions referred to as “computer programs.” The computer programs typically include one or more instructions, when read and executed by one or more processors in a computer (or a computing device), cause the computer to perform operations to execute elements involving the various aspects of the invention. The program code or one or multiple program modules may cause a processing unit (e.g., any processor unit or module, such as a single- or multi-core processor, a multi-processor unit, and so on) of a user device to perform a method for assisting in easing hardship of falling asleep, based on at least one embodiment as illustrated in FIG. 10, 11, or 12. The program code or one or multiple program modules can be implemented as or embedded into application software, system software, a mobile app, a web app, without limited thereto. The non-transitory readable information storage medium in each of the embodiments can be exemplified as, without limitation to, an optical information storage medium, a magnetic information storage medium, or a memory, such as a memory card, firmware, ROM or RAM.

As provided in the various embodiments above, sleep guidance is provided by the sleep assistant system through the user device, based on at least one biosignal detected from the user. The sleep guidance by the sleep assistant system can assist the user in developing awareness of one's sleep problem can be resolved by self-adjustment, e.g., changing their behavior and practicing sleep hygiene (e.g., as provided in advices of the sleep guidance) and following the directions the user to ease the falling-asleep hardship by self-adjustment (e.g., as provided in training or directions from the sleep guidance). The user thus can ease the hardship of falling asleep, not merely relying on external adjustments (e.g., environment adjustments). In some embodiment, assistance arrangement can be performed, including one or more types of sleep guidance and/or environmental condition control, based on the falling-asleep hardship index, so as to reduce the falling-asleep hardship for a user before the user falls asleep.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims which are to be accorded with the broadest interpretation so as to encompass all such modifications and similar structures.

What is claimed is:

1. A sleep assistant system comprising:
a first user device including at least one sensor for sensing at least one biosignal from a user;
a second user device, able to communicate with the first user device to receive data indicating the at least one biosignal, for providing, based on the received data, sleep guidance in visual form and/or audio form to assist the user before falling asleep in changing a falling-asleep hardship index for the user from a first state to a second state, wherein the sleep guidance is a portion of user interaction between the second user device and the user;
wherein the second user device determines the falling-asleep hardship index for the user based on the at least one biosignal; the falling-asleep hardship index determined before the sleep guidance is provided is at the first state; the falling-asleep hardship index determined after the sleep guidance is provided is at the second state; and the second state, different from the first state, indicates a less hardship for falling asleep in terms of the at least one biosignal from the user.

2. The sleep assistant system according to claim 1, wherein the second user device determines, based on the first state, that the sleep guidance includes directions in visual form and/or audio form to instruct the user to follow the directions to adjust one's respiration rate before falling asleep.

3. The sleep assistant system according to claim 1, wherein the second user device determines, based on the first state, that the sleep guidance includes directions in visual form and/or audio form to instruct the user to follow the directions to practice meditation before falling asleep.

4. The sleep assistant system according to claim 1, wherein the second user device determines, based on the first state, that the sleep guidance includes an advice about sleep, wherein the advice is presented in visual form and/or audio before falling asleep.

5. The sleep assistant system according to claim 1, wherein the second user device determines, based on the second state, that the second user device provides additional sleep guidance which includes directions in visual form and/or audio form to instruct the user to follow the directions to adjust one's respiration rate before falling asleep.

6. The sleep assistant system according to claim 1, wherein the second user device determines, based on the second state, that the second user device provides additional sleep guidance which includes directions in visual form and/or audio form to instruct the user to follow the directions to practice meditation before falling asleep.

7. The sleep assistant system according to claim 1, wherein the second user device determines, based on the second state, that the second user device provides additional sleep guidance which includes an advice about sleep, wherein the advice is presented in visual form and/or audio before falling asleep.

8. The sleep assistant system according to claim 1, wherein the second user device adjusts the sleep guidance or provides additional sleep guidance based on data received from the first user device during the sleep guidance.

9. The sleep assistant system according to claim 1, wherein the second user device sends at least one control signal, based on the received data, to adjust environmental condition for the user so as to assist the user before falling asleep in changing the falling-asleep hardship index to a state indicates a less hardship for falling asleep in terms of the at least one biosignal from the user.

10. The sleep assistant system according to claim 9, further comprising:
a plurality of environment adjusting devices operative based on the at least one control signal to adjust the environmental condition for the user;
wherein the environmental condition includes one or more of light condition, sound condition, temperature condition, and air quality condition.

11. The sleep assistant system according to claim 1, wherein the at least one biosignal includes one or more of a heartbeat signal, a body temperature signal, a blood pressure signal, a respiration rate signal, and a skin conductivity signal.

12. The sleep assistant system according to claim 1, wherein the second user device comprises:
a processing unit, communicating with the first user device, for receiving the data indicating the at least one biosignal and determining the falling-asleep hardship index for the user based on the received data; and
a display unit, wherein the processing unit, for providing, based on the received data, sleep guidance in visual form through the display unit and/or audio form to assist the user before falling asleep in changing the falling-asleep hardship index for the user from the first state to the second state.

13. The sleep assistant system according to claim 1, wherein the first user device is a wearable device able to communicate with the second user device electrically or wirelessly.

14. A method for assisting in easing hardship of falling asleep comprising:
receiving data indicating at least one biosignal;
determining a falling-asleep hardship index based on the received data to indicate hardship of falling asleep for a user; and
providing, based on the falling-asleep hardship index, sleep guidance in visual form and/or audio form to assist the user before falling asleep in changing the falling-asleep hardship index for the user from a first state to a second state, wherein the sleep guidance is a portion of user interaction between the second user device and the user;
wherein the falling-asleep hardship index determined before the sleep guidance is provided is at the first state; the falling-asleep hardship index determined after the sleep guidance is provided is at the second state; and the second state, different from the first state, indicates a less hardship for falling asleep in terms of the at least one biosignal from the user.

15. The method according to claim 13, wherein the sleep guidance is determined based on the first state and includes directions in visual form and/or audio form to instruct the user to follow the directions to adjust one's respiration rate before falling asleep.

16. The method according to claim 13, wherein the sleep guidance is determined based on the first state and includes directions in visual form and/or audio form to instruct the user to follow the directions to practice meditation before falling asleep.

17. The method according to claim 13, wherein the sleep guidance is determined based on the first state and includes an advice about sleep, wherein the advice is presented in visual form and/or audio before falling asleep.

18. The method according to claim 13, further comprising:
providing additional sleep guidance which includes directions in visual form and/or audio form to instruct the user to follow the directions to adjust one's respiration rate before falling asleep, wherein the additional sleep guidance is determined based on the second state.

19. The method according to claim 13, further comprising:
providing additional sleep guidance which includes directions in visual form and/or audio form to instruct the user to instruct the user to follow the directions to practice meditation before falling asleep, wherein the additional sleep guidance is determined based on the second state.

20. The method according to claim 13, further comprising:
providing additional sleep guidance which includes an advice about sleep, wherein the advice is presented in visual form and/or audio before falling asleep.

21. The method according to claim 13, wherein the sleep guidance is adjusted or additional sleep guidance is provided, based on the data received during the sleep guidance.

22. The method according to claim 13, further comprising:
sending at least one control signal, based on the received data, to adjust environmental condition for the user so as to assist the user before falling asleep in changing the falling-asleep hardship index to a state indicates a less hardship for falling asleep in terms of the at least one biosignal from the user.

23. The method according to claim 21, wherein the environmental condition includes one or more of light condition, sound condition, temperature condition, and air quality condition.

24. The method according to claim 13, wherein the at least one biosignal includes one or more of a heartbeat signal, a body temperature signal, a blood pressure signal, a respiration rate signal, and a skin conductivity signal.

25. A non-transitory computer-readable medium, having stored thereon instructions, which when executed by a processing unit, cause the processing unit to perform:
receiving data indicating at least one biosignal;
determining a falling-asleep hardship index based on the received data to indicate hardship of falling asleep for a user; and
providing, based on the falling-asleep hardship index, sleep guidance in visual form and/or audio form to assist the user before falling asleep in changing the falling-asleep hardship index for the user from a first state to a second state, wherein the sleep guidance is a portion of user interaction between the second user device and the user;
wherein the falling-asleep hardship index determined before the sleep guidance is provided is at the first state; the falling-asleep hardship index determined after the sleep guidance is provided is at the second state; and the second state, different from the first state, indicates a less hardship for falling asleep in terms of the at least one biosignal from the user.
